(19) United States
(12) Patent Application Publication
(10) Pub. No.: US 2023/0414564 A1
(43) Pub. Date: Dec. 28, 2023

(54) BLUE LIGHT OXIDATION INHIBITOR AND SCREENING METHOD FOR SAME

(71) Applicant: SHISEIDO COMPANY, LTD., Chuo-ku, Tokyo (JP)

(72) Inventors: Katsuhiko TSUCHIDA, Tokyo (JP); Natsuki SAKIYAMA, Tokyo (JP)

(73) Assignee: SHISEIDO COMPANY, LTD., Chuo-ku, Tokyo (JP)

(21) Appl. No.: 18/036,618

(22) PCT Filed: Nov. 29, 2021
(86) PCT No.: PCT/JP2021/043696

§ 371 Date: May 11, 2023

(30) Foreign Application Priority Data
Dec. 11, 2020 (JP) 2020-206296

(51) Int. Cl. A61K 31/375; A23L 33/175; A23L 33/15; A61K 31/185; G01N 33/15

(52) U.S. Cl. CPC A61K 31/375; A23L 33/175; A23L 33/15; A61K 31/185; G01N 33/15

(57) ABSTRACT

Provided is a chemical agent effective for inhibiting blue light-induced oxidation, and a screening method for the same. A blue light oxidation inhibitor comprising one or more compounds selected from the group consisting of compounds including the structure of formula I, L(+)-ascorbic acid compounds and pantetheine-S-sulfonic acid-containing compounds is effective for inhibiting oxidation induced by blue light.

### FIELD

The present invention relates to a chemical agent that efficiently inhibits oxidation induced by exposure to blue light, and to a screening method for the same.

### BACKGROUND

With ever wider usage of digitalized monitor devices in recent years, incidence of exposure to blue light has been increasing and is predicted to further increase in the future. Blue light is also one of the components of sunlight. Blue light has been reported to have various adverse effects on skin (PTLs 1 and 2, NPLs 1 and 2), reaching more deeply into the skin due to its longer wavelength compared to UV light. There remains a need for strategies to protect skin using methods that counteract blue light.

In this context, PTL 1 discloses a composition comprising vitamin B6 or a derivative thereof for protection of human skin from blue light, and a method of coating human skin with the composition. PTL 2 discloses an inhibitor of blue light-induced cell growth having bilberry extract as an active ingredient. However, further research is necessary to discover new means for efficiently inhibiting the adverse effects of blue light.

### CITATION LIST

### Patent Literature

[PTL 1] Japanese Patent Public Inspection No. 2020-512307
[PTL 2] Japanese Unexamined Patent Publication No. 2015-44773
[PTL 3] Japanese Unexamined Patent Publication No. 2019-120704

### Non Patent Literature

[NPL 1] K. Dong et. al, Blue light disrupts the circadian rhythm and create damage in skin cells, Int J Cosmet Sci. 2019 December; 41(6):558-562. doi: 10.1111/ics.12572.
[NPL 2] Y Nakashima et al, Blue light-induced oxidative stress in live skin, Free Radic Biol Med. 2017 July; 108:300-310. doi: 10.1016
[NPL 3] Ou-Yang, H. et. al., A chemiluminescence study of UVA-induced oxidative stress in human skin in vivo. J. Invest. Dermatol. 122, 1020-1029 (2004).
[NPL 4] Prasad, A. & Pospisil, P., Ultraweak photon emission induced by visible light and ultraviolet A radiation via photoactivated skin chromophores: in vivo charge coupled device imaging, J. Biomed. Opt. 17, 085004 (2012).
[NPL 5] Tsuchida, K., Visualization of UV-induced oxidative stress using biophoton imaging, Fragrance Journal, 2020 July; 17-21.

### SUMMARY

### Technical Problem

It is an object of the present invention to provide a chemical agent that inhibits oxidation induced by exposure to blue light, and to a screening method for the same.

### Solution to Problem

The present inventors have discovered a chemical agent that efficiently inhibits oxidation induced by exposure to blue light, as determined by UPE measurement, and to a screening method for the same. Upon much additional research on a large variety of materials based on this discovery, it was found that specific components, among which are hypotaurine, thiotaurine, L(+)-ascorbic acid compounds and calcium pantetheine-S-sulfonate, exhibit effects that inhibit oxidation by blue light, and the present invention was completed.

The present application encompasses the following invention:
(1) A blue light oxidation inhibitor comprising a substance that efficiently inhibits lipid oxidation induced by exposure to blue light, wherein the inhibition of oxidation is detectable using UPE as the index.
(2) The inhibitor according to (1), wherein the lipid is one or more lipids selected from among linoleic acid, oleic acid, linolenic acid, squalene, palmitoleic acid and phospholipids.
(3) The inhibitor according to (1) or (2), wherein the component that inhibits oxidation is a compound including the structure of formula I, an L(+)-ascorbic acid compound, or a compound including pantetheine-S-sulfonic acid.
(4) The inhibitor according to (3), wherein the compound including the structure of formula I is hypotaurine or thiotaurine.
(5) The inhibitor according to (3), wherein the L(+)-ascorbic acid compound is L(+)-ascorbic acid, sodium L(+)-ascorbate or calcium L(+)-ascorbate.
(6) An inhibitor of optical oxidation of lipids, which comprises a compound including pantetheine-S-sulfonic acid.
(7) The inhibitor according to (6), wherein the optical oxidation is oxidation by UVA or blue light.
(8) The inhibitor according to any one of (3), (6) or (7), wherein the compound including pantetheine-S-sulfonic acid is calcium pantetheine-S-sulfonate or sodium pantetheine-S-sulfonate.
(9) A blue light oxidation inhibitor comprising one or more compounds selected from among hypotaurine, thiotaurine, L(+)-ascorbic acid and calcium pantetheine-S-sulfonate.
(10) The chemical agent according to any one of (1) to (9), wherein the lipid is a skin component, an eye component or a food or beverage component.
(11) A blue light oxidation inhibitor that efficiently inhibits lipid oxidation induced by exposure to blue light, comprising a compound including the structure of formula I.
(12) A screening method for a blue light oxidation inhibitor, wherein the effect of inhibiting lipid oxidation induced by exposure to blue light measured by UPE is used as the index.
(13) An examination method for a blue light oxidation inhibitor, wherein the method comprises:
a step in which a lipid is contacted with a candidate substance;
a step in which the lipid contacted with the candidate substance and the same lipid not contacted with the substance are exposed to blue light;
a step in which the UPE levels of the lipids exposed to the blue light are measured; and
a step in which it is determined that the candidate substance is a blue light oxidation inhibitor if the UPE level measured from the lipid contacted with the candidate substance is lower than that measured from the lipid not contacted with the candidate substance.
(14) The method according to (11), wherein the lipid is one or more lipids selected from among linoleic acid, oleic acid, linolenic acid, squalene, palmitoleic acid and phospholipids.

### Advantageous Effects of Invention

According to the invention there is provided a chemical agent effective for inhibiting blue light-induced oxidation, and a screening method for the same. According to the invention it is possible to devise a countermeasure against oxidation induced by blue light.

### BRIEF DESCRIPTION OF DRAWINGS

FIG. 1 shows an example of photon counting data for UPE after irradiating a linoleic acid mixture with blue light.

FIG. 2 shows the rate of change in UPE induced after irradiating blue light onto linoleic acid mixtures with addition of different samples.

FIG. 3 shows the rate of change in UPE induced after irradiating blue light and UVA onto linoleic acid mixtures with addition of different samples.

FIG. 4 shows the rate of change in UPE induced after irradiating blue light and UVA onto oleic acid mixtures with addition of 5% hypotaurine.

FIG. 5 shows an example of UPE imaging data after coating a portion of human skin with 5% hypotaurine and irradiating with blue light. The 5% hypotaurine was coated in the area within the dotted circle.

FIG. 6 shows the results of antioxidant evaluation using an antioxidant measurement kit with samples at different concentrations (5%, 1%, 0.2%), as DPPH radical scavenging rates (%).

FIG. 7 shows results of peroxide number measurement [meq/kg] after irradiating blue light and UVA irradiation onto linoleic acid with addition of plant extract A or B.

### DESCRIPTION OF EMBODIMENTS

### [Blue Light Oxidation Inhibitor]

One aspect of the invention relates to a blue light oxidation inhibitor comprising a substance that efficiently inhibits oxidation induced by exposure to blue light (also to be referred to as “oxidation induced by blue light”, “oxidation by blue light” or “blue light oxidation”),
wherein the inhibition of oxidation is detectable using UPE as the index.

The term “oxidation” refers to oxidation of various substances including lipids such as linoleic acid, oleic acid, linolenic acid, squalene, palmitoleic acid and phospholipids. Lipids are a class of biological components found in animals such as humans. Biological components include skin components which make up skin, and eye components, examples of which are lipids such as linoleic acid, oleic acid, linolenic acid, squalene, palmitoleic acid and phospholipids, proteins such as collagen and elastin, and DNA and melanin precursors (DHICA). Oxidation of skin components such as lipids is known to produce various adverse effects including skin spots, wrinkles, sagging, reduced skin elasticity and skin aging. Oxidation of melanin precursors is known to result in darkening of the skin. Lipids are also present in the eye, and oxidation of these lipids is known to cause the eye condition known as cataract. Food and beverage components in foods and beverages include abundant amounts of lipids, and oxidation of those lipids is known to result in adverse effects for the foods or beverages.

Oxidation of substances can be measured by methods such as an active oxygen removal test, i.e. a DPPH radical scavenging test, and evaluation of peroxide number. The state of oxidation can be measured by detection of Ultraweak Photon Emission (UPE). UPE, also referred to as biophotons, consists of weak emission from the body or biological components, and it is believed to be a cause of oxidation reaction. For example, the state of oxidation caused by ultraviolet rays can be measured by detecting UPE, as described in PTL 3 and NPLs 4 and 5.

Components are known that inhibit oxidation, and they include many types of antioxidants such as vitamins, coenzyme Q and polyphenols. However, little research has yet been conducted on the effectiveness of these antioxidants on different types of oxidation, and it was unexpected that the inhibiting effect against oxidation by light differs depending on the wavelength by the which the oxidation is induced. The present inventors have found, surprisingly, using UPE as an index of oxidation, that several antioxidants have different inhibiting effects against oxidation by UV light and against oxidation by blue light. Based on this knowledge, the present inventors further found that the inhibiting effect against oxidation by blue light is higher than the inhibiting effect against oxidation by light of other wavelengths such as UV light, and have discovered substances that efficiently inhibit the oxidation induced by exposure to blue light. The inhibiting effect against oxidation with focus on blue light has been difficult to measure by other means, such as by evaluating radical scavenging power as demonstrated in the Examples.

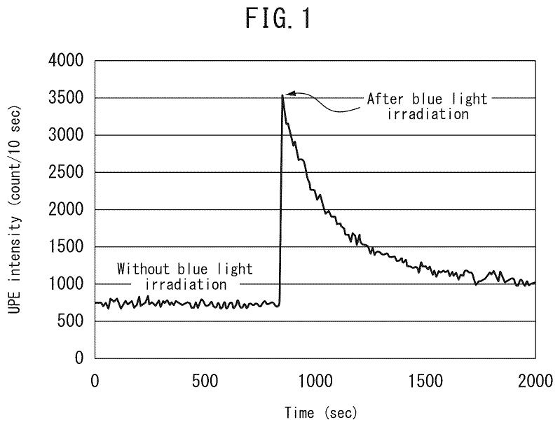

The UPE used as an index of oxidation according to the invention is extremely weak spontaneous emission generated by the body and by components such as lipids, proteins, amino acids, DNA and/or melanin precursors, and as described in PTL 3 and NPL 5, it can be measured using an optical detector equipped with a detecting unit such as a high-sensitivity, low-noise, cooled CCD camera or photomultiplier tube (PMT) which allows detection of ultraweak UPE. One example of an optical detector is a weak luminescence intensity detector (CLA-IDFsk by Tohoku Electronic Industrial Co.). The wavelength of the emitted light to be detected differs depending on the detector (cooled CCD camera or photomultiplier tube), but for measurement of UPE induced by blue light or UV light the device is preferably able to detect wavelengths of 300 to 1300 nm. With an optical detector using a photomultiplier tube, it is possible to obtain UPE induced by blue light or UV light as photon counting data such as shown in FIG. 1. The UPE level can be calculated as the integrated value for the UPE count number within a specific time period after irradiation with blue light or UV light, which may be a specific time period from 1000 seconds, 1500 seconds or 2000 seconds after irradiation until the UPE returns to the level without irradiation, as shown in FIG. 1. In addition to in vivo methods of measuring emission from the body, the UPE can also be measured by in vitro or ex vivo measurement of emission from samples of biological components, among which are skin components or eye components including lipids such as linoleic acid, oleic acid, linolenic acid, squalene, palmitoleic acid and phospholipids, proteins such as collagen and elastin, amino acids, DNA and/or melanin precursors, or food or beverage components included in foods or beverages, skin models, tissue sections surrounding skin or eye, cells, and foods or beverages. An “eye component” for the purpose of the invention includes the concept of tissue components inside or surrounding the eye, which includes not only the eye bulb but also muscles such as the extraocular muscles surrounding the eye bulb or ciliary muscles, and fat including orbital fat, aqueous humor, lacrimal fluid and Meibomian glands.

Blue light is light in the visible light region of 400 nm to 500 nm. Blue light has a longer wavelength than UV light which includes UVA (320 to 400 nm) and UVB (280 to 320 nm), and therefore reaches deeper into the skin. Blue light is also known to have various adverse effects on skin, including skin cell growth inhibition (PTLs 1 and 2, NPLs 1 and 2). It is therefore important to protect skin from blue light. With the increasing usage of monitor devices in recent years there are also increasing concerns regarding oxidation of the eyes or foods by blue light.

According to the invention, a “substance that efficiently inhibits oxidation induced by exposure to blue light” (also “blue light oxidation-inhibiting substance”) is a substance wherein the UPE level measured from a component such as a lipid that has been exposed to blue light, after contact with the candidate substance, is lower than when the component has not been contacted, and according to one embodiment it refers to a substance with a higher inhibiting effect against oxidation by blue light, as compared with its inhibiting effect against oxidation by UV light such as UVA or UVB.

The phrase “the UPE level measured from a component . . . after contact with the candidate substance, is lower than when the component has not been contacted” means that after having been contacted with the candidate substance, the UPE level measured from the component is reduced by a statistically significant difference at a significance level of 5% (Wilcoxon rank sum test or t test, for example), or reduced by 5% or greater, 10% or greater, 20% or greater, 30% or greater, 40% or greater, 50% or greater, 60% or greater, 70% or greater, 80% or greater, 90% or greater or 100% reduction, for example, compared to when it has not been contacted with the candidate substance (control).

For a component such as a lipid has been exposed to an oxidation-inducing factor such as blue light or UV light, the oxidation inhibiting effect is the percentage (%) of reduction in UPE level measured from the component that has been contacted with the substance, with respect to the UPE level measured from the component that has not been contacted with the substance, and it is represented by the following formula 1.

[Mathematical Formula 1]

Oxidation inhibiting effect (%)=100−((UPE level with contact)/(UPE level without contact))×100 Formula I:

The phrase “higher inhibiting effect against oxidation by blue light, as compared with its inhibiting effect against oxidation by UV light” means, for example, that the oxidation inhibiting effect (%) based on UPE measured from the component that has been exposed to blue light, is higher than the oxidation inhibiting effect (%) based on UPE measured from the component that has been exposed to UV light, with a statistically significant difference at a significance level of 5% (by Wilcoxon rank sum test or t test, for example), or alternatively, higher by 5% or greater, 10% or greater, 20% or greater, 30% or greater, 40% or greater, 50% or greater, 60% or greater, 70% or greater, 80% or greater, 90% or greater, 100% or greater or 200% or greater, or even greater, for example.

An example of a “blue light oxidation-inhibiting substance” according to the invention is the group of compounds represented by formula I below.

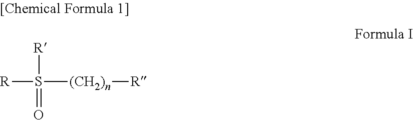

(where R is OH, SH, F, Cl or NH₂,
R′ is 0 of absent (and absent when R is, —OH),
R″ is OH, SH, F, CL, or NH₂, and
n is, an integer, including 0)

Examples of compounds of formula I include hypotaurine (CAS Accession No.: 300-84-5) and thiotaurine (CAS Accession No.: 2937-54-4). Hypotaurine and taurine are substances produced by cysteine metabolism, with cysteine being converted to hypotaurine and then to taurine. The function of hypotaurine was therefore predicted to be similar to that of taurine or cysteine. However, the present inventors found that, while little of the inhibiting effect against blue light oxidation according to the invention was exhibited by taurine or cysteine, a high inhibiting effect against blue light oxidation was observed with hypotaurine and thiotaurine, the inhibiting effect against blue light oxidation being higher than the inhibiting effect against oxidation by UVA.

Other “blue light oxidation-inhibiting substances” are compounds including L(+)-ascorbic acid compounds and pantetheine-S-sulfonic acid.

Examples of the L(+)-ascorbic acid compounds of the invention include L(+)-ascorbic acid (CAS Accession No.: 50-81-7), sodium L(+)-ascorbate (CAS Accession No.: 134-03-2) and calcium L(+)-ascorbate (CAS Accession No.: 5743-27-1). According to one aspect, the L(+)-ascorbic acid compound of the invention is any one or more compounds selected from the group consisting of L(+)-ascorbic acid, sodium L(+)-ascorbate and calcium L(+)-ascorbate.

Ascorbic acid compounds such as L(+)-ascorbic acid and ascorbic acid glucoside are known as antioxidants and skin whiteners. It was therefore predicted that both L(+)-ascorbic acid and ascorbic acid glucoside would exhibit similar inhibiting effects against blue light oxidation. However, the present inventors found that, while little of the blue light oxidation-inhibiting effect of the invention was observed with ascorbic acid glucoside, a high blue light oxidation-inhibiting effect was observed with L(+)-ascorbic acid.

Examples of compounds including pantetheine-S-sulfonic acid are salts such as calcium pantetheine-S-sulfonate and sodium pantetheine-S-sulfonate.

Calcium pantetheine-S-sulfonate (CAS Accession No.: 34644-00-3) is known as a skin whitener, but it is not known to have an optical oxidation-inhibiting effect. Nevertheless, the present inventors found that calcium pantetheine-S-sulfonate exhibits an optical oxidation-inhibiting effect, including a high blue light oxidation-inhibiting effect.

Moreover, the blue light oxidation-inhibiting effects of thiotaurine and calcium pantetheine-S-sulfonate were discovered using UPE as the index, but could not be detected by evaluation based on radical scavenging power, as demonstrated in the Examples.

### [Optical Oxidation Inhibitor]

One aspect of the invention relates to an optical oxidation inhibitor comprising an optical oxidation-inhibiting substance such as a compound including pantetheine-S-sulfonic acid.

Optical oxidation is oxidation of a substance such as a lipid by light of various wavelengths, such as UV light and/or blue light. The optical oxidation may be oxidation by light including UVA and/or blue light, or oxidation by light consisting of UVA and/or blue light.

An optical oxidation-inhibiting substance is a substance that, when a component such as a lipid is exposed to light such as blue light or UV light, lowers the UPE level measured from the component that has contacted with the substance, compared to the component without such contact.

Using UPE as the index, the present inventors have found that compounds that include pantetheine-S-sulfonic acid, such as calcium pantetheine-S-sulfonate, inhibit oxidation of substances including skin components such as lipids by optical oxidation, i.e. oxidation by blue light or UV light.

The optical oxidation-inhibiting effect of calcium pantetheine-S-sulfonate was first discovered using UPE as the index, whereas it could not be detected by evaluation based on radical scavenging power, as demonstrated in the Examples.

The blue light oxidation inhibitor and optical oxidation inhibitor of the invention will collectively be referred to herein as “agent of the invention”.

### [Composition]

The agent of the invention may be prepared as a composition in combination with one or more other components, such as an excipient, carrier and/or diluent. The constitution and form of the composition may be as desired, and it may be appropriately selected depending on conditions including the active ingredient and the purpose of use. The composition may be prepared by a common method as a formulation in an appropriate combination with an excipient, carrier and/or diluent or other components, depending on the dosage form.

For example, the agent of the invention may be added to a composition such as a cosmetic, quasi drug, pharmaceutical, functional food or other food or beverage for the purpose of preventing or improving oxidation of skin components, eye components or food or beverage components such as lipids, proteins, amino acids, DNA or melanin precursors, by light such as blue light or UV, preventing or improving skin spots, loss of skin clarity, wrinkles, shrinkage, sagging, aging or eye conditions such as cataracts, caused by oxidation of skin components, eye components or food or beverage components such as lipids, proteins, amino acids, DNA or melanin precursors by light such as blue light or UV, improving skin color, skin elasticity, skin function, resistance, or eye function, or preventing oxidation of foods or beverages or lengthening their storage life. For addition to a cosmetic, addition may be to any cosmetic that is to be applied to skin, such as a sunscreen, cosmetic water, essence, beauty cream or aftercare lotion, or it may be added to an external preparation for skin that can be directly applied to skin. Alternatively, it may be added to a pharmaceutical or quasi drug that is an ophthalmic agent, external preparation for skin or oral drug. The agent of the invention may have optional compounding ingredients added as necessary and appropriate, such as are commonly used in compositions such as cosmetics, pharmaceuticals, quasi drugs and foods or beverages, within ranges that do not interfere with its effect. Examples of optional compounding ingredients include oils, surfactants, powders, coloring materials, water, alcohols, thickeners, chelating agents, silicones, humectants, aromatics, drug components, antiseptic agents, pH adjustors, neutralizing agents, excipients, coloring agents, binders, disintegrators, dispersing agents, stabilizers and gelling agents. For example, the agent may contain other drug components, antioxidants, preservatives, ultraviolet scattering agents or ultraviolet absorbers that promote oxidation inhibiting effects for lipids or skin components against light of other wavelengths.

When the invention is to be applied in a cosmetic, pharmaceutical, quasi drug or food or beverage, the type, purpose, form and manner of use of the agent of the invention or its composition is not restricted and may be determined as appropriate, so long as the effect of the invention is not impeded. For example, the active ingredient of the agent of the invention, as one or more compounds selected from the group consisting of compounds with the structure of formula I, such as hypotaurine or thiotaurine, and pantetheine-S-sulfonic acid-containing compounds such as calcium pantetheine-S-sulfonate or sodium pantetheine-S-sulfonate, may be added in any desired proportion such as 0.0001 to 0.001 wt %, 0.001 to 0.005 wt %, 0.005 to 0.01 wt %, 0.01 to 0.02 wt %, 0.02 to 0.05 wt %, 0.05 to 0.1 wt %, 0.1 to 1.0 wt %, 1.0 to 10 wt %, 10 to wt % or 50 to 100 wt %, for example, with respect to the entire cosmetic.

The form of the agent or composition of the invention are not limited to the dosage forms mentioned above, however.

### [Screening Method]

One aspect of the invention relates to a screening method for a blue light oxidation inhibitor, wherein the effect of inhibiting oxidation induced by exposure to blue light measured by UPE is used as the index.

Another aspect of the invention relates to an examination method for a blue light oxidation inhibitor, wherein the method comprises:
a step in which a lipid is contacted with a candidate substance such as a skin component, eye component or food or beverage component;
a step in which the lipid contacted with the candidate substance and the lipid not contacted with the substance are exposed to blue light;
a step in which the UPE levels of the lipids exposed to the blue light are measured; and
a step in which it is determined that the candidate substance is a blue light-specific oxidation inhibitor if the UPE level measured from the lipid contacted with the candidate substance is lower than that measured from the lipid not contacted with the candidate substance.

With the screening method or examination method of the invention it is possible to select whether or not a candidate drug has a blue light oxidation-inhibiting effect, and to propose new product development, skin care, eye disease prevention and food or beverage storage means.

### [Method of Inhibiting Blue Light Oxidation and Optical Oxidation]

The present invention provides a method of inhibiting skin damage by optical oxidation by blue light or UV by administration of an agent or composition of the invention. According to one aspect, the skin damage is due to oxidation of skin components such as lipids by blue light oxidation or optical oxidation. The method of the invention may be for the purpose of beautifying, instead of treatment by a doctor or health care professional.

The present invention also provides a method of inhibiting eye damage by optical oxidation by blue light or UV by administration of an agent or composition of the invention. According to one aspect, the eye damage is due to oxidation of eye components such as lipids by blue light oxidation or optical oxidation.

The target of administration of the agent or composition of the invention may be a subject found to have objective or subjective damage to skin or eyes by optical oxidation by blue light or UV, or a subject desiring to prevent damage to skin or eyes by such oxidation. For example, the subject may be one judged to have a high UPE level induced by light such as UV light or blue light, or a high degree of oxidation of the skin or eyes, or a subject who cannot avoid exposure to blue light present in sunlight or produced when using devices such as PCs, tablets or smartphones, or a subject who is concerned about or desiring to prevent skin damage such as skin spots, loss of skin clarity, wrinkles, shrinkage, sagging, reduced skin elasticity or skin aging, or eye damage such as cataract, caused by UV light or blue light.

The present invention also provides a method of inhibiting damage to foods or beverages by optical oxidation by blue light or UV, by the use of an agent or composition of the invention. According to one aspect, the damage to a food or beverage is due to oxidation of food or beverage components such as lipids by blue light oxidation or optical oxidation. The method of the invention may also include addition of the agent or composition of the invention during production of a food or beverage. A food or beverage according to the invention may be a food or beverage that is prone to degradation by optical oxidation of food or beverage components such as lipids by blue light or UV, such as a frozen food, a retort pouch food, seasoning, a bottled food or beverage or a PET bottle beverage, for example.

### EXAMPLES

The present invention will now be explained in greater detail by examples. However, the invention is in no way limited by the Examples.

### Preparation of Candidate Substances

The following substances were used as candidate substances.

TABLE 1

| | |
|---|---|
| L(+)-Ascorbic acid | CAS Accession No.: 50-81-7 |
| L-Ascorbic acid glucoside | CAS Accession No.: 129499-78-1 |
| Thiotaurine | CAS Accession No.: 2937-54-4 |
| Hypotaurine | CAS Accession No.: 300-84-5 |
| Pantetheine-S-calcium sulfonate | CAS Accession No.: 34644-00-3 |
| Taurine | CAS Accession No.: 107-35-7 |
| L-Cysteine | CAS Accession No.: 52-90-4 |
| *Extract A | Plant A extract |
| *Extract B | Plant B extract |

The candidate substances listed in Table 1 were added to ultrapure water to prepare 5 wt % aqueous solutions as samples.

The samples were prepared by mixing the substances in the following compositions with linoleic acid and oleic acid as biological components. As a control, ultrapure water was used instead of a 5% aqueous solution of the candidate substance.

TABLE 2

| | | |
|---|---|---|
| Linoleic acid or oleic acid | 1000 | μL |
| 1,3-Butylene glycol | 950 | μL |
| 5% aqueous solution of candidate substance | 50 | μL |
| Total | 2000 | μL |

### Example 1: Determining Oxidation Degree by UPE Count Method (In Vitro)

A 2 mL portion of the sample prepared by the method described above was added to a plastic container and irradiated with 6 mW/cm² blue light (430 nm LED, CL-H1-430-9-1, Asahi Spectra Co., Ltd.) using an LED light source (CL-1501, Asahi Spectra Co., Ltd.), for a period of 10 minutes. The experiment was conducted while shielded, to exclude the effects of light of other wavelengths. For the control, ultrapure water was added instead of the starting material aqueous solution.

The irradiated sample was transferred to a quartz glass container and set in the black box of a photon counter (Tohoku Electronic Industrial Co.). Photon counting of UPE was initiated one minute after irradiation was complete. UPE was detected by a built-in photomultiplier tube (PMT, R2257P, Hamamatsu Photonics, K.K.) in the photon counter, with measurement being conducted while shielded.

The integrated value of UPE intensity for 300 seconds after the start of UPE measurement was determined and the rate of change in UPE was calculated by formula 2, to evaluate the oxidative stress inhibiting effect.

[Mathematical Formula 2]

Rate of change in UPE=(UPE intensity of material added sample−UPE intensity of control sample (non-irradiated))−(UPE intensity of control sample−UPE intensity of control sample (non-irradiated)) Formula 2:

An experiment was carried out for L(+)-ascorbic acid, thiotaurine, hypotaurine and calcium pantetheine-S-sulfonate, which exhibited high blue light oxidation-inhibiting effects, in the same manner as above except that irradiation was with an UVA lamp (300 to 400 nm: 3.5 mW/cm²) [TOREX FL20SBL, Toshiba Medical Supply] instead of blue light (430 nm LED: 6 mW/cm²).

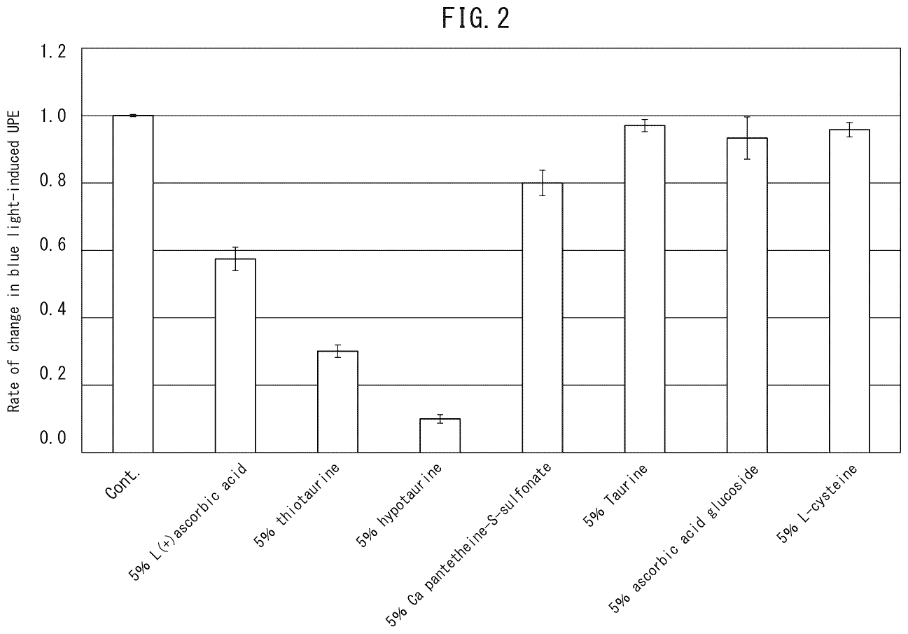

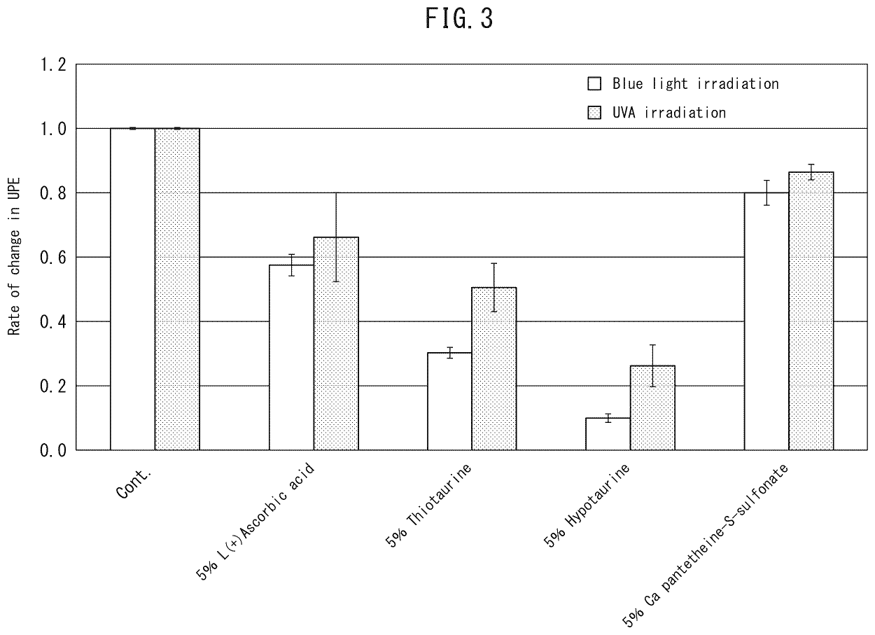

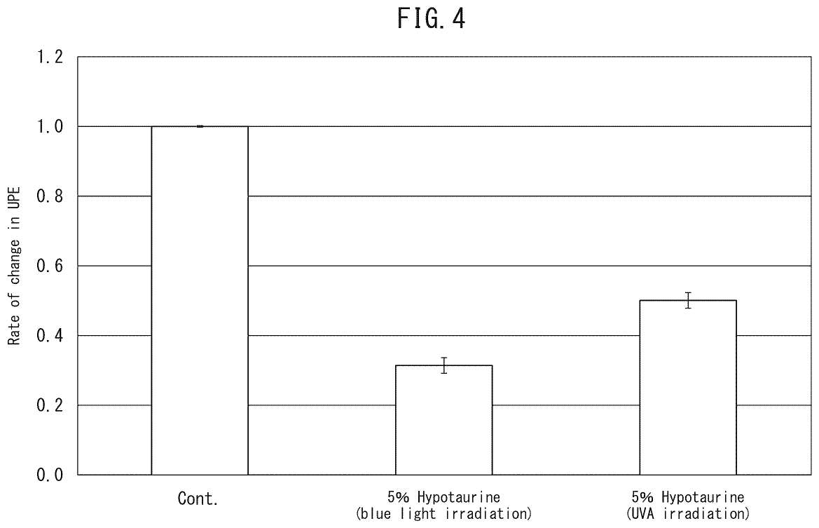

FIG. 2 shows the rate of change in blue light-induced UPE by the candidate substance. FIG. 3 shows both the rate of change in blue light-induced UPE and the rate of change in UV light-induced UPE. Based on these graphs, the oxidation inhibiting effects differ depending on the type of candidate substance. Especially notable is the difference in the rate of change in blue light-induced UPE and the rate of change in UV light-induced UPE. The results demonstrated that L(+)-ascorbic acid, thiotaurine, hypotaurine and calcium pantetheine-S-sulfonate have excellent blue light oxidation-inhibiting effects. Taurine, L-ascorbic acid glucoside and L-cysteine, on the other hand, were not found to have significant blue light oxidation-inhibiting effects. The results for oleic acid are likewise shown in FIG. 4. It is seen that the effect of the invention is effective with oleic acid as well as with linoleic acid.

### Example 2: Measurement of Oxidative Stress by UPE Imaging

A 20 μL aqueous solution of 5% hypotaurine was applied to the center of the stratum corneum side of human skin tissue (KAC Co., dorsal region), and allowed to stand for 40 minutes while shielded.

After irradiation with blue light (430 nm LED, 6 mW/cm²) for 10 minutes, the hypotaurine aqueous solution was wiped off and immediately observed by UPE imaging using a high-sensitivity cooled CCD camera (850S CCD42-40, Spectral Instruments Inc.), while shielded (photographing time: 10 minutes).

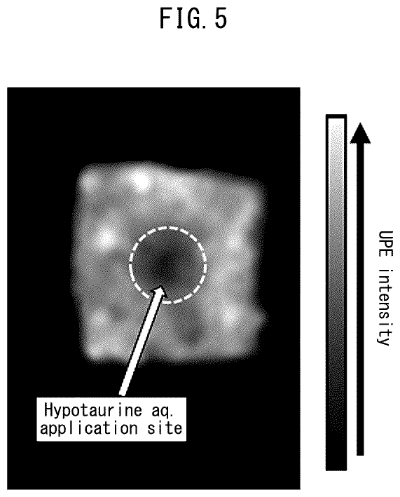

The results are shown in FIG. 5. The UPE intensity was found to be lowered by the extent of hypotaurine application. In the non-applied regions, UPE intensity was high and increased under blue light. This indicates that the excellent blue light oxidation-inhibiting effect seen in the in vitro experiment of Example 1 is also observed in experimentation with actual skin.

### Comparative Example 1: Evaluation of Radical Scavenging Rate

Methods for measuring oxidation inhibiting effects by radical scavenging rate evaluation are known. The present inventors therefore examined oxidation inhibiting effects of the aforementioned candidate substances at different concentrations, by the following procedure using a DPPH Antioxidant Assay Kit (Dojindo Laboratories).

The candidate substances of Example 1 were used as 5 wt %, 1 wt % and 0.2 wt % aqueous solutions. Blank 1, Blank 2, Sample and Sample Blank Solution listed in Table 3 were prepared by the manufacturer's protocol described in general below, and the radical scavenging rates were calculated.

TABLE 3

| | Blank 1 | Blank 2 | Sample | Sample Blank |
|---|---|---|---|---|
| Sample solution | — | — | 20 μl | 20 μl |
| Water | 20 μl | 20 μl | — | — |
| Ethanol | — | 100 μl | — | 100 μl |
| Assay Buffer | 80 μl | 80 μl | 80 μl | 80 μl |
| DPPH working | 100 μl | — | 100 μl | — |
| solution | | | | |

1. Each candidate substance was loaded into a 96 well plate as a 5 wt %, 1 wt % or 0.2 wt % aqueous solution.
2. Water was added to Blank 1 and Blank 2 at 20 μl each.
3. Assay Buffer was added at 80 μl to each well.
4. After adding 100 μl of ethanol to Blank 2 and the Sample blank, it was thoroughly mixed by pipetting.
After adding 100 μl of DPPH working solution to the sample and Blank1, it was thoroughly mixed by pipetting.
6. The plate was incubated for 30 minutes at 25° C. in a dark environment.
7. The absorbance at 517 nm was measured with a plate reader (Spectramax 250, Molecular Devices).
8. The radical scavenging rate (%) of each sample was calculated by the following formula 3.

[Mathematical Formula 3]

Radical scavenging rate (%)=(ACS−AS/ACS)×100 Formula 3:

### (ACS=Blank1−Blank2, AS=Sample−Sample Blank)

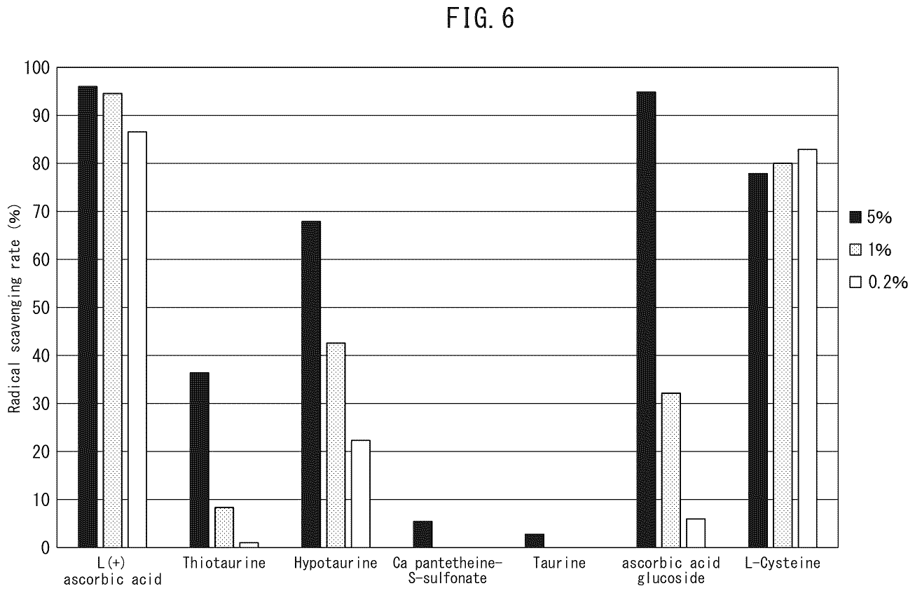

The results are shown in FIG. 6. FIG. 6 shows that L(+)-ascorbic acid, hypotaurine, L-ascorbic acid glucoside and L-cysteine have high radical scavenging power. High radical scavenging power was not observed with thiotaurine, taurine and calcium pantetheine-S-sulfonate, however. Whereas thiotaurine and calcium pantetheine-S-sulfonate both exhibited excellent blue light oxidation-inhibiting effects in Example 1, blue light oxidation-inhibiting effects were not measurable as oxidation inhibiting effects based on DPPH radical scavenging power. A blue light oxidation-inhibiting substance was therefore found to be present, which was undetectable based on DPPH radical scavenging power. For calcium pantetheine-S-sulfonate as well, an optical oxidation-inhibiting effect including a UV oxidation inhibiting effect was found using UPE as the index, whereas it was not detectable based on DPPH radical scavenging power.

### Comparative Example 2: Evaluation by Peroxide Number

Plant extracts were evaluated by peroxide number according to the following procedure.

First, samples with the compositions listed in the table were prepared.

TABLE 4

| | | |
|---|---|---|
| Linoleic acid | 13.2 | mL |
| 1,3-Butylene glycol | 1.65 | mL |
| Plant extract | 0.15 | mL |
| Total | 15 | mL |

Next, 15 mL of prepared sample was added to a glass beaker and irradiated for 3 hours with blue light (430 nm LED: 6 mW/cm²) or UVA lamp (300 to 400 nm: 1.2 mW/cm²), while shielded. As a control, the same amount of 1,3-butylene glycol was added instead of plant extract. The peroxide number was then measured by the acetic acid-chloroform method described below.

To 5 g of irradiated test sample there was added 30 ml of a liquid mixture of chloroform and acetic acid (chloroform:acetic acid=2:3), and the components were dissolved. After nitrogen exchange, 0.5 ml of a saturated potassium iodide solution was added and reaction was conducted for 5 minutes at room temperature in a dark environment. Following the reaction, 30 ml of water was added and the mixture was shaken. A 0.01 mol/L sodium thiosulfate standard solution was then used for titration (indicator: 1% starch solution), the peroxide number (meg/kg) was calculated by the following formula 4.

[Mathematical Formula 4]

Peroxide number ((meg/kg))=(titer of 0.01 mol/L sodium thiosulfate standard solution)×titer (mL)×(10/5 (g)) Formula 4:

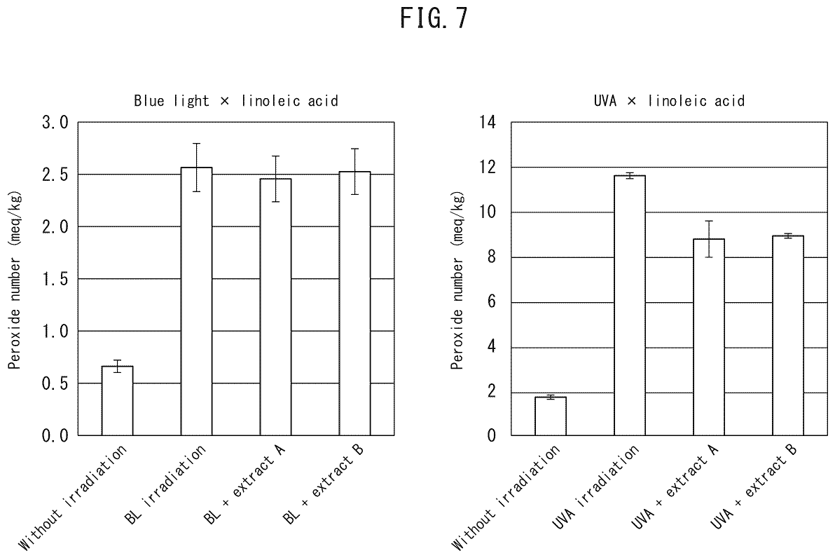

The results are shown in FIG. 7. The UVA oxidation inhibiting effect was higher than the blue light oxidation-inhibiting effect with the extracts of plant A and plant B, with no notable blue light oxidation-inhibiting effect seen when using these extracts. This result also suggested that the blue light oxidation-inhibiting effect and UVA oxidation inhibiting effect differ depending on the substance.

The results described above demonstrated that by using UPE as the index it is possible to search for blue light oxidation inhibitors including substances that efficiently inhibit lipid oxidation induced by exposure to blue light. High blue light oxidation-inhibiting effects were found with compounds including the structure of formula I, L(+)-ascorbic acid compounds and pantetheine-S-sulfonic acid-containing compounds. Pantetheine-S-sulfonic acid-containing compounds were also found to have optical oxidation-inhibiting effects. Such substances inhibit damage to skin, eyes and foods or beverages induced by blue light or UV light, and can consequently prevent or improve skin damage such as wrinkles, shrinkage, sagging, reduced skin elasticity and skin aging, or eye damage such as cataract, and oxidation of foods or beverages, which is caused by blue light or UV light.

1. A method for inhibiting blue light oxidation in a subject in need thereof, comprising administering a substance that efficiently inhibits lipid oxidation induced by exposure to blue light to the subject,
wherein the inhibition of oxidation is detectable using UPE as the index.

2. The method according to claim 1, wherein the lipid is one or more lipids selected from among linoleic acid, oleic acid, linolenic acid, squalene, palmitoleic acid and phospholipids.

3. The method according to claim 1, wherein the component that inhibits oxidation is a compound including the structure of formula I, an L(+)-ascorbic acid compound, or a compound including pantetheine-S-sulfonic acid.

4. The method according to claim 3, wherein the compound including the structure of formula I is hypotaurine or thiotaurine.

5. The method according to claim 3, wherein the L(+)-ascorbic acid compound is L(+)-ascorbic acid, sodium L(+)-ascorbate or calcium L(+)-ascorbate.

6. An inhibitor of optical oxidation of lipids, which comprises a compound including pantetheine-S-sulfonic acid.

7. The inhibitor according to claim 6, wherein the optical oxidation is oxidation by UVA or blue light.

8. The inhibitor according to claim 6, wherein the compound including pantetheine-S-sulfonic acid is calcium pantetheine-S-sulfonate or sodium pantetheine-S-sulfonate.

9. A blue light oxidation inhibitor comprising one or more compounds selected from among hypotaurine, thiotaurine, L(+)-ascorbic acid and calcium pantetheine-S-sulfonate.

10. The method according to claim 1, wherein the lipid is a skin component, an eye component or a food or beverage component.

11. A blue light oxidation inhibitor that efficiently inhibits lipid oxidation induced by exposure to blue light, comprising a compound including the structure of formula I.

12. A screening method for a blue light oxidation inhibitor, wherein the effect of inhibiting lipid oxidation induced by exposure to blue light measured by UPE is used as the index.

13. An examination method for a blue light oxidation inhibitor, wherein the method comprises:
a step in which a lipid is contacted with a candidate substance;
a step in which the lipid contacted with the candidate substance and the same lipid not contacted with the substance are exposed to blue light;
a step in which the UPE levels of the lipids exposed to the blue light are measured; and
a step in which it is determined that the candidate substance is a blue light oxidation inhibitor if the UPE level measured from the lipid contacted with the candidate substance is lower than that measured from the lipid not contacted with the candidate substance.

14. The method according to claim 11, wherein the lipid is one or more lipids selected from among linoleic acid, oleic acid, linolenic acid, squalene, palmitoleic acid and phospholipids.

15. The inhibitor according to claim 7, wherein the compound including pantetheine-S-sulfonic acid is calcium pantetheine-S-sulfonate or sodium pantetheine-S-sulfonate.

16. The inhibitor according to claim 6, wherein the lipid is a skin component, an eye component or a food or beverage component.

17. The inhibitor according to claim 7, wherein the lipid is a skin component, an eye component or a food or beverage component.

18. The inhibitor according to claim 8, wherein the lipid is a skin component, an eye component or a food or beverage component.

19. The inhibitor according to claim 9, wherein the lipid is a skin component, an eye component or a food or beverage component.
